(19) United States
(12) Patent Application Publication
(10) Pub. No.: US 2004/0033268 A1
(43) Pub. Date: Feb. 19, 2004

(54) Medicinal composition and in particular its use in fluid therapy

(76) Inventors: Pascal Gustin, Brussels (BE); Carole Cambier, Eghezee (BE); Thierry Clerbaux, Brussels (BE); Albert Frans, Messancy (BE); Bruno Detry, Ittre (BE)

Correspondence Address: VENABLE, BAETJER, HOWARD AND CIVILETTI, LLP, P.O. BOX 34385, WASHINGTON, DC, 20043-9998, US

(21) Appl. No.: 10/398,780

(22) PCT Filed: Oct. 16, 2001
(86) PCT No.: PCT/BE01/00180

(30) Foreign Application Priority Data
Oct. 16, 2000 (EP) 00203619.2

(51) Int. Cl. A61K 35/14

(52) U.S. Cl. 424/529

(57) ABSTRACT

The invention concerns a medicinal composition comprising, in therapeutically efficient amounts, hypertonic sodium chloride, at least a molecule directly or indirectly providing a relaxing effect on the vascular smooth muscles, and at least a molecule capable of supplying an exogenous phosphate input.

The present invention relates to a novel medicinal composition and in particular to its use in fluid therapy.

In cattle, toxi-infectious pathologies, including neonatal diarrhoea and endotoxin shock, require such a treatment.

With regard to neonatal diarrhoea, it is commonly accepted that the first treatment to be set up is oral or parenteral fluid therapy, according to the clinical state of the animal. However, current treatments are aimed solely at re-establishing the water and ion balance, re-establishing the blood pH and covering any energy deficit. Up to the present time, the treatment normally advised in the scientific literature dealing with parenteral fluid therapy in cattle is based on the administration of large volumes of isotonic crystalloid solutions, the quantities perfused being calculated according in particular to the degree of dehydration and acidosis of the animal. In order to re-establish the circulating volume, other authors also recommend the administration of small volumes of a hypertonic saline solution, with dextran added or not. According to the literature, these saline solutions must be accompanied by the administration of an alkalising agent in order to re-establish the blood pH. It is therefore clear that, at the present time, disorders involving tissue oxygenation existing in a diarrhoeic calf are never taken into account in evaluating the efficacy of the treatment. More seriously, in some cases, current treatments even aggravate these problems with tissue oxygenation. This is because, for example, the massive administration of bicarbonates with a view to re-establishing the pH may cause relative alkalosis and hypochloremia, thus promoting an increase in the affinity of the haemoglobin for oxygen and making the transportation of oxygen by the blood less efficient.

With regard to endotoxin shock, it is also accepted that the institution of treatment by parenteral fluid therapy is a priority. However, this is aimed essentially at reestablishing the circulating volume. The scientific literature describes the use of various perfusion solutions, namely: isotonic crystalloid solutes, hypertonic crystalloid solutes (whether or not with a colloid such as dextran added), but also plasma and whole blood. The effects of these various treatments on the transportation of oxygen by the haemoglobin and the oxygenation of the tissues have not always been studied in depth.

Moreover, the patents U.S. Pat. No. 5,238,684, 5,236,712, 5,147,650, 5,089,477 and 4,981,687 describe formulations, intended to be administered preferably orally, which aim to increase the physiological response to exercise, notably via an increase in the cardiac output. These solutions contain water, electrolytes, glycerol and an additional energy source. These solutions in no way aim to manipulate the transportation of oxygen by the haemoglobin, nor to improve the tissue oxygenation.

The aim of the present invention is to develop a novel medicinal composition. This must be able to combat the tissue hypoxia which arises during toxi-infectious pathologies, in mammals, in particular in ruminants, such as cattle, and including human beings. In the course of these pathological processes, the invention must make it possible to maintain, in the beings afflicted, their consumption of oxygen by means in particular of an increase in the quantity of oxygen extracted at the tissues, whilst combining this original mechanism with an increase in the cardiac output.

In order to resolve this problem, there is provided, according to the invention, a medicinal composition comprising, in therapeutically effective quantities, hypertonic sodium chloride, at least one molecule directly exerting an effect of relaxation of the vascular smooth muscles, and at least one molecule able to supply an exogenous phosphate input. This composition comprises a therapeutic “tripod” necessary for guaranteeing the therapeutic success sought. It acts by a combination of original action mechanisms allowing a better extraction of oxygen at the tissue level, namely a reduction in the intrinsic affinity of haemoglobin for oxygen, an in vivo increase in the transportation of the oxygen by the haemoglobin and an increase in the intrinsic capacity of the tissues to extract oxygen. The better extraction of oxygen at the tissue level avoids the development of tissue hypoxia.

According to one embodiment of the invention, the composition also comprises an alkalinising agent able to increase the blood pH within a range compatible with a suitable transportation of oxygen. This alkalinising agent in suitable quantity enables the relative acidosis to be maintained. As alkalinising agents, bicarbonates, acetates, propionates and lactates, in particular sodium bicarbonate, can be mentioned amongst others.

According to one advantageous form of the invention, the composition consists of a combination of a first perfusion solution and a second perfusion solution, the first perfusion solution comprising water, the said hypertonic sodium chloride and the said at least one molecule exerting a relaxation effect, and the second perfusion solution comprising water and the said at least one molecule able to supply an exogenous phosphate input. By virtue of this embodiment, administered by perfusion, for example intravenously, it is possible to increase the cardiac output, which reinforces the phenomenon of oxygen extraction at the tissue level.

Such a formulation allows separate manufacture and storage of the components as well as possibly a chronologically separate administration of the two solutions, according to the speed of action of the components envisaged. Moreover, it also allows, if necessary, a simultaneous perfusion of the two solutions at two different points on the body to be treated and an application of these two solutions which is staged over time.

According to the invention, the sodium chloride is at a concentration of 5% to 10% in the first perfusion solution. Advantageously, use will be made of doses of approximately 5 to 10 ml/kg of body weight. Molecule exerting a relaxation effect on the smooth muscles can be understood to mean, according to the invention, vasodilators in general and caffeine in particular. Molecules able to provide an exogenous phosphate input can be taken to mean according to the invention an inorganic phosphate, for example sodium phosphate, or an organic phosphate such as a triphosphate, for example adenosine triphosphate, or diphosphoglycerate, as well as in general any substance capable of modifying the intra-erythrocyte content of diphosphoglycerate.

According to a particular form of the invention, the second perfusion solution also contains at least one component intended to correct the water, electrolytic and energy balance. A component of this type can be taken to mean sodium chloride, potassium chloride, glucose etc.

Other medicinal compositions according to the invention are indicated in the claims given hereinafter, as well as the use of these compositions for the manufacture of a medicine intended for the therapeutic or prophylactic treatment of hypoxic tissue disorders, in particular those arising during toxi-infectious pathologies, in mammals.

The invention will now be described in detail by means of examples given below non-limitingly.

### EXAMPLE 1

Under normal conditions, the transportation of oxygen in dissolved form represents only a small proportion (approximately 1%) of the total transportation. For the remainder, the oxygen is conveyed to the tissues after fixing on haemoglobin.

At the pulmonary level, the haemoglobin is saturated with oxygen. In the tissues at rest, approximately one third of the total quantity thus fixed diffuses in the interstitial liquid, the remainder returning to the lungs. The fixing of the oxygen on haemoglobin depends above all on the partial oxygen pressure (PO₂) in the plasma.

Conventionally, in all mammals, the factors regulating the affinity of oxygen for haemoglobin are the blood pH, the body temperature, the carbon dioxide partial pressure and the degree of fixing of 2,3-diphosphoglycerate (2,3 DPG) on the haemoglobin.

These mechanisms allow exchanges of oxygen in the organism. In the arterial blood, the oxygen partial pressure is high, the temperature and the CO₂ contents decrease, whilst the pH increases, all conditions favourable to the fixing of the oxygen on the haemoglobin. In the venous blood and in the tissues, on the other hand, the oxygen partial pressure is low, the pH is decreasing, the temperature and CO₂ contents are increasing, all factors favourable to the release of oxygen.

During a study whose purpose was to investigate tissue oxygenation disorders in diarrhoeic calves, considering the organism no longer in its entirety but in a vital corporeal territory in particular, it has been possible to demonstrate that an adaptation of this oxygenation by an increase in the extraction of oxygen at the tissue level did not exist in all organs. The ability of the tissues to extract oxygen even appeared to be diminished. It appeared that, if the exchanges occurring between the arterial blood and the jugular venous blood are considered, diarrhoeic calves suffer from an inability to extract the oxygen which is carried to them by the blood. This inability can be explained by:

an increase in the affinity of the oxygen for haemoglobin, illustrated by the decrease in the oxygen partial pressure at 50% saturation of haemoglobin measured under standard conditions (pH: 7.4; PCO₂: 40 mm of Hg; temperature: 37° C.), hereinafter referred to as the P50 standard (P50 std). This phenomenon is explained in particular by a reduction in the level of 2,3 DPG;

hypothermia and hypocapnia, which oppose the positive effects of acidosis on the transportation of oxygen by the blood, as illustrated by the fall in partial oxygen pressures at 50% saturation of haemoglobin in the arterial compartments (P50a) and venous compartments (P50v);

an intrinsic inability of the tissues to extract the oxygen which is carried to them, as illustrated by the increase in the partial oxygen pressure at the venous level (PvO₂) in diarrhoeic calves.

Consequently in the present example a series of treatments of diarrhoeic calves by a conventional method was carried out, with a commercially available product, with various components of a composition according to the invention and with a composition according to the invention, for purposes of comparison.

In all the following examples, the doses are calculated with respect to 1 kg of body weight.

a) The conventional solution tested is an isotonic crystalloid solution of sodium chloride (40 ml/kg), glucose (20 ml/kg) and sodium bicarbonate, to be perfused. The quantity of sodium bicarbonate to be added is calculated as follows: number of mmoles of bicarbonate to be added=Weight (kg)×basic deficit in the venous blood (mmoles/l)×0.6 (1/kg), where 0.6 represents the volume of distribution of the bicarbonate in the extracellular liquid compartment. Perfusion rate: 50 ml/kg/h for the first hour, and then 20 ml/kg/h.

b) The commercial solution, Lactetrol^{R}, is a medicine indicated for the correction of dehydration, whether or not accompanied by metabolic acidosis, in cattle. It is important to note that this product is one of the rare medicines available on the market for the treatment, by parenteral fluid therapy, of symptoms associated with neonatal diarrhoea. The formula of the specialty is as follows:

| | | |
|---|---|---|
| Sodium chloride | 5.76 | mg |
| Potassium chloride | 0.37 | mg |
| Calcium chloride | 0.37 | mg |
| Magnesium chloride | 0.2 | mg |
| Sodium lactate | 5.04 | mg |
| Methyl parahydroxybenzoate | to 1 | ml |
| Water for injection | | |

Volume to be administered: 40 ml/kg. Perfusion rate: 10 ml/kg/h.

c) A composition consisting of two perfusion solutions:

hypertonic solution A (NaCl 7.5%, 5 ml/kg, 1 ml/kg/min): this solution being at the limit of the osmolarity permitted in treatment, the elements aimed at re-establishing the ion balance and maintaining the water balance and the pH are contributed separately via a solution B.

isotonic solution B (glucose: 20 ml/kg and sodium bicarbonate administered in sufficient quantity to return the basic deficit to zero). This solution can if necessary be supplemented by other ions (K⁺, Mg⁺⁺ etc). Administration rate: 50 ml/kg/h for the first hour and then 20 ml/kg/h.

The quantity of sodium bicarbonate to be added is calculated as described above.

d) A composition consisting of two perfusion solutions:

hypertonic solution A (NaCl 7.5%, 7.5 ml/kg with 0.4 g of caffeine added per litre; 1 ml/kg/min): this solution being at the limit of the osmolarity permitted in treatment, the elements aimed at re-establishing the ion balance and maintaining the water balance and the pH are contributed separately via a solution B.

solution B (isotonic NaCl+glucose: 10 g/l+potassium chloride: 1 g/l: 40 ml/kg and isotonic sodium bicarbonate administered in sufficient quantity to bring the pH to 7.2). This solution can if necessary be supplemented by other ions (Mg⁺⁺ etc). Administration rate: 20 ml/kg/h.

The quantity of sodium bicarbonate to be supplied was calculated according to the Henderson-Hasselbalch equation.

e) A composition made up as follows:

isotonic sodium chloride+glucose: 10 g/l+potassium chloride: 1 g/l: 40 ml/kg, and isotonic inorganic phosphates (NaH₂PO₄.H₂O: 2.45 g/l, Na₂HPO₄.2H₂O: 19.82 g/l): 16 ml/kg. This solution can if necessary be supplemented with other ions (Mg⁺⁺ etc). Administration rate: 27.5 ml/kg/h.

f) Composition according to the invention consisting of two perfusion solutions:

solution A:

Non-pyrogenic sterile water

Hypertonic NaCl (7.5%): 75 g/l

Caffeine: 0.4 g/l

(7.5 ml/kg; perfusion rate: 1 ml/kg/min)

solution B:

non-pyrogenic sterile water

Isotonic buffer solution of inorganic phosphates (NaH₂PO₄.H₂O: 2.45 g/l, Na₂HPO₄.2H₂O: 19.82 g/1): 16 ml/kg; perfusion rate: 25 ml/kg/h.

With a view to re-establishing the blood volume, correcting the electrolytic balance and maintaining the pH within limits compatible with the transportation of oxygen (7.2), various compounds can be added to this solution, namely: NaCl, KCl, MgCl₂, glucose, NaHCO₃.

For example: solution of isotonic sodium chloride+glucose 10 g/l+potassium chloride 1 g/l (40 ml/kg) and isotonic sodium bicarbonate if necessary (pH<7.2).

Each of these compositions was tested on a sample of approximately 10 diarrhoeic calves. It should be noted that, in the case of the compositions formed from two solutions, solution B was perfused 15 minutes after the start of the perfusion of solution A.

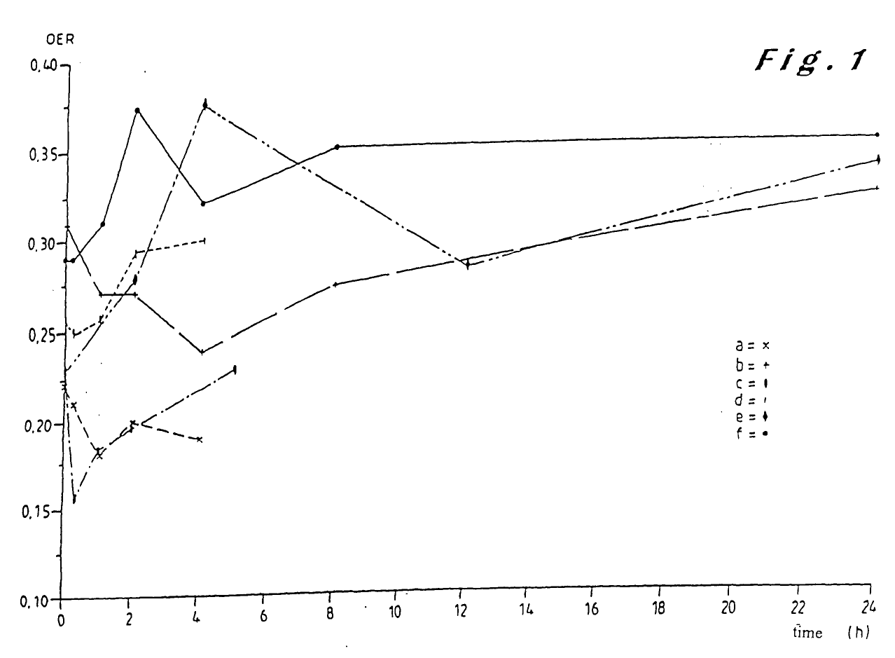

FIG. 1 illustrates the change over time in the quantity of oxygen extracted at the tissue level (OER) following the administration of each of compositions a) to f). OER is the ratio between the difference in the oxygen content in the arterial blood (CaO₂) and venous blood (CvO₂) and the oxygen content in the arterial blood. In practice the arterial oxygen content and the venous oxygen content are calculated as follows:

CaO₂=Hb×BO₂×[SaO₂/100]+(α×PaO₂)

CvO₂=Hb×BO₂×[SvO₂/100]+(α×PvO₂)

where Hb represents the level of haemoglobin in the animal, BO₂ represents the quantity of oxygen fixed by the haemoglobin (1.39 mlO₂/g Hb) and α represents the coefficient of solubility of the oxygen (0.003 ml.100 ml⁻¹. mm Hg⁻¹).

The proportion of oxygen extracted at the tissue level is then expressed as a ratio: OER=(CaO₂−CvO₂)/CaO₂.

From this FIG. 1 it can be deduced that the conventional treatment a) and the Lactetrol solution b) are not capable of increasing the extraction of oxygen at the tissue level.

Even though a favourable effect of the chloride ion on the transportation of the oxygen in vivo had been remarked, in healthy calves (Cambier et al, Effects of Hyperchloremia on Blood Oxygen Binding in Healthy Calves, The American Physiological Society, 1998, p. 1267-1272), this treatment (composition c)) unfortunately proved incapable of increasing the extraction of oxygen at the tissue level in diarrhoeic calves, this parameter even having a tendency to decrease. The results obtained in vivo in healthy calves can therefore not be transposed to sick calves.

Finally, there appears with composition d) a slight increase in the quantity of oxygen extracted, which nevertheless does not prove significant, whilst composition e) and the composition according to the invention f) exhibit a significant and lasting increase therein.

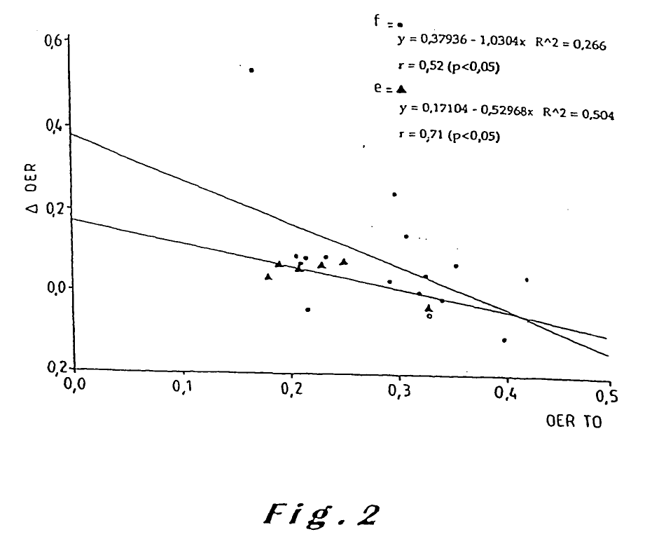

However, the advantage of the composition according to the invention f) compared with composition e) appears when the variations in the OER recorded during treatment are compared with the initial values of OER measured at TO (FIG. 2). A significant negative correlation is observed with the two treatments. This means that the animals having the most marked deficiencies from the point of view of the OER derive the greatest benefit from the treatments. However, compared with the two solutions e) and f), it appears that the increases in OER obtained with composition e) are less than those obtained with the composition according to the invention f), as shown by the ordinates giving rise to the equations for the linear regressions relating to these two treatments (respectively 0.17 for composition e) and 0.38 for the composition according to the invention f)).

In addition, it must be emphasised that only the composition according to the invention f) involves all the mechanisms allowing an increase in the extraction of oxygen at the tissue level. This phenomenon may prove to be very important under pathological conditions, one mechanism being able to supplement the other in the event of deficiency.

For the record, the extraction of oxygen at the tissue level can be improved via three mechanisms:

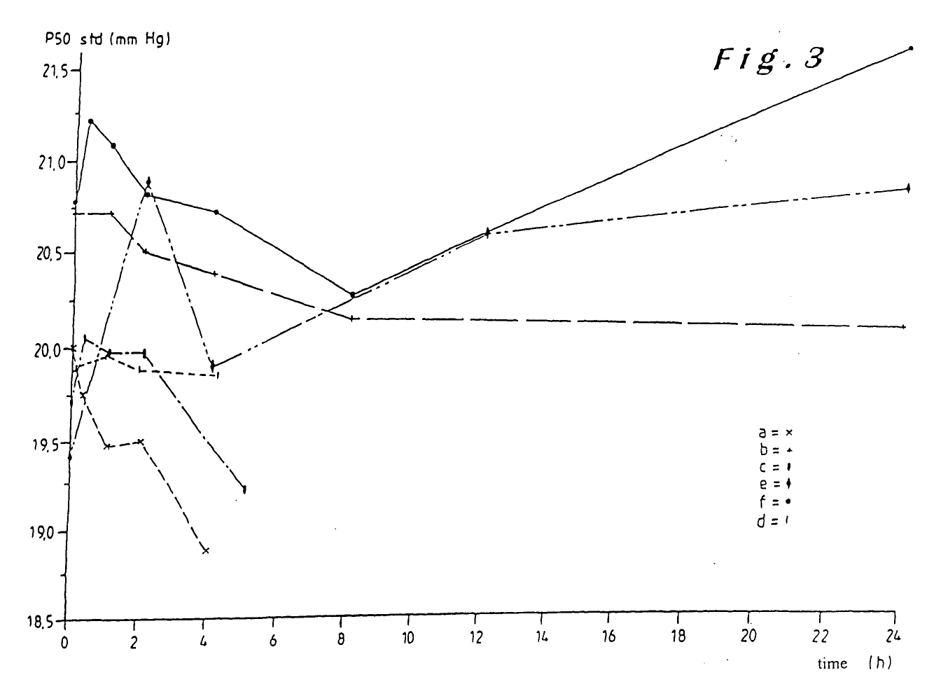

1. through a decrease in the intrinsic affinity of haemoglobin for oxygen. This phenomenon is illustrated by an increase in the P50 std. FIG. 3 shows a significant increase in the P50 std when composition e) is administered and when the composition according to the invention f) is administered whilst no significant effect is obtained, or even deleterious effect (reduction in the P50 standard) are recorded, with the other treatments.

2. by an in vivo modification of the factors controlling the transportation of the oxygen by the blood, and in particular the pH. The beneficial effects recorded, in vitro, on the P50 std (cf point 1) can thus be increased or decreased in animals. In vivo, the capacity for transportation of oxygen by the blood is evaluated by calculating the partial oxygen pressures P50 a and P50 v. Ideally, an increase in the P50 a and in the P50 v is required, by reinforcing, via in particular variations in pH, the effect on the P50 std.

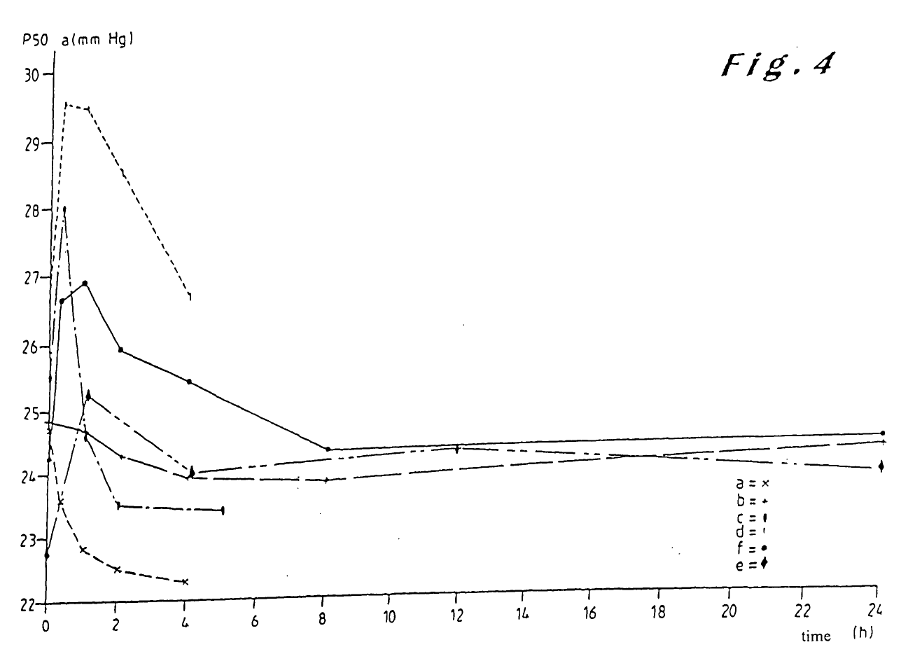

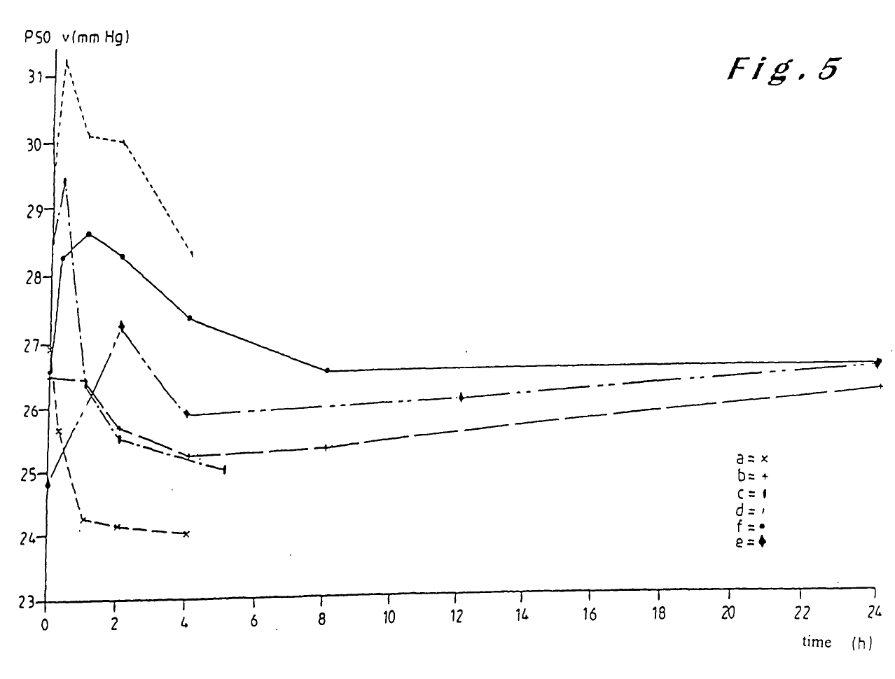

As illustrated by FIGS. 4 and 5, in vivo, composition e) and the composition according to the invention f) improve the oxygen transportation capacities. However, with regard to composition e), this effect has its origin mainly in the reduction in the intrinsic affinity of the haemoglobin for oxygen, illustrated by the increase in the P50 std. This phenomenon is only slightly reinforced in vivo. Thus, at t=2 hours, if composition e) increases the P50 std by 8%, the P50 a and P50 v are increased only by respectively 10% and 9%, showing the absence of any intervention of other mechanisms favourable to the release of oxygen at the tissue level. In vivo, the intervention of these mechanisms regulating the oxygen transportation may prove to be very important in animals where the P50 standard increases only slightly following the perfusion. Thus the effects obtained on the P50 standard with composition e) are variable. For proof, at t=2 hours, the increase in P50 standard varies from 4% to 13% according to the individual, and only 33% of the animals treated with composition e) maintain an increase in P50 standard for 24 hours. In these animals, recourse to other regulating mechanisms would be necessary. Unlike solution e), the composition according to the invention f) allows, in addition to the increase in the P50 standard, the development of hyperchloremia and the maintenance of a relative acidosis, two matters reinforcing the effects on the P50 standard and favourable to the release of oxygen at the tissue level. The weight of these various mechanisms varies over time. Thus, at t=1 hour, the composition according to the invention f) causes an increase in the P50 standard of only 1% but increases the P50 a and P50 v respectively by 11% and 8%, because of the development of hyperchloremia and the maintenance of a relative acidosis. At t=24 hours, the balance between these two functions reverses since the role played by the modifications to the intrinsic affinity of haemoglobin for oxygen becomes preponderant;

3. by an increase in the ability of the tissues to extract oxygen, marked by a decrease in the oxygen partial pressure at the venous level (PvO₂).

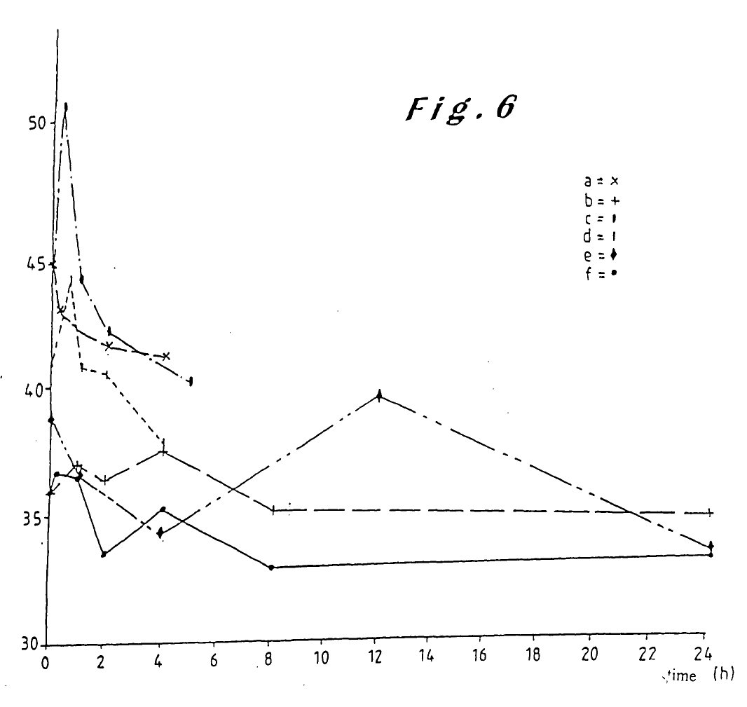

As illustrated by FIG. 6, the hypertonic composition c) significantly increases the PvO₂. The administration of a vasodilator simultaneously with a hypertonic solution (composition d)) or in the absence of a hypertonic solution, as is the case with composition e), cancels out this effect. By combining a vasodilator, a hypertonic solution of NaCl and phosphates, according to the composition according to the invention f), the inherent ability of the tissues to extract oxygen is optimal and is maintained throughout the treatment.

The methods of determining the PvO₂ and the P50 are described for example in C. Cambier et al., American Journal of Veterinary Research, 61, 2000, p. 299-304.

In addition to these three mechanisms acting in the increase in the extraction of oxygen at the tissue level, a perfusion solution can also induce beneficial effects via haemodynamic modifications (increase in cardiac output).

According to the data in the literature, and on the basis of our own experiencing in calves afflicted by endotoxin shock (cf Example 3), hypoxia may result in:

an impairment in the supply of oxygen by the blood (DO₂) resulting in particular from haemodynamic disturbance (reduction in cardiac output),

a decrease in the extraction of oxygen at the tissue level, which has just been discussed.

These two mechanisms are combined to various degrees in individuals afflicted by toxi-infectious pathologies in order to result in the hypoxic states noted clinically. It is therefore vital to have available therapeutic solutions for improving all these functions, by all possible mechanisms. The solution according to the invention, in addition to its effects on the OER, improves the D0₂ because of the increase in the cardiac output related to the water input but in particular to a hyperosmotic effect in the intravascular compartment. FIG. 8 illustrates the increase in the D0₂ recorded in calves suffering from endotoxin shock and treated with the composition according to the invention.

The administration of an isotonic solution, such as solution e), may cause an increase in the cardiac output because of the water input but this effect is limited and deferred in time compared with the almost immediate and significant effects of the hypertonic solutions, such as the composition according to the invention f).

In summary, in addition to the fact of exerting a greater original effect on the OER (cf FIG. 2) than solution e), the solution according to the invention f) therefore acts on all the mechanisms liable to combat disorders involving the extraction of oxygen at the tissue level.

In addition, it exerts immediate and significant haemodynamic effects through its direct but also hyperosmotic effects.

### EXAMPLE 2

A comparative test was carried out on three samples of diarrhoeic calves. The first sample (n=12) was subjected to the conventional treatment a) described in Example 1, the second sample (n=18) to Lactetrol and the third sample (n=16) to the composition according to the invention f) of Example 1.

A clinical score for the treated calves was then established by analysing the symptoms of the illness and integrating evaluations such as the reduction in the suction reflex, the slowing down in the threat reflex, the decrease in tactile sensitivity, the decrease in the ability to stand upright, etc. The score is 0 for healthy calves and 13 in a comatose animal. The results of this evaluation are set out in Table 1 below.

TABLE 1

| Parameters/ | Invention | Conventional Trt | Lactetrol Trt |
|---|---|---|---|
| Time (h) | (n = 16) | (n = 12) | (n = 18) |
| Clinical score | | | |
| 0 | 3.6 ± 0.6 | 5.1 ± 1.2 | 4.6 ± 0.8 |
| 0.25 | 3.3 ± 0.5 | 4.9 ± 1.2 | N.D. |
| 1 | 2.6 ± 0.4**†Δ | 4.9 ± 1.1 | 4.5 ± 0.8 |
| 2 | 1.7 ± 0.3***†Δ | 4.3 ± 1.1 | 3.9 ± 0.7* |
| 4 | 1.3 ± 03***†ΔΔ | 3.3 ± 0.9* | 3.6 ± 0.8** |
| 8 | 0.9 ± 0.2**ΔΔ | N.D. | 2.9 ± 0.6** |
| 24 | 1.4 ± 03***Δ | N.D. | 3.6 ± 0.8* |

N.D.: not determined
Trt: treatment
The values are expressed as follows: averages ± SE.
*: value significantly different from the value recorded at T0 in the same group (*: p < 0.05, **: p < 0.01, ***: p < 0.001)
†: value significantly different from the value recorded at the same time in the conventional treatment group (†: p < 0.05)
Δ: value significantly different from the value recorded at the same time in the Lactetrol^{R} treatment group (Δ: p < 0.05, ΔΔ: p < 0.01)

### EXAMPLE 3

Systemic disorders in the extraction of oxygen at the tissue level having been revealed during endotoxin shock, a comparative test was carried out on two samples of healthy calves who had received an intravenous injection of endotoxins of Escherichia coli serotype 055:B5, 0.2 μg/kg, at time −30 minutes. At time 0, a first sample (n=3) was subjected to treatment with a composition according to the invention and a second sample (n=3) was not subjected to any treatment.

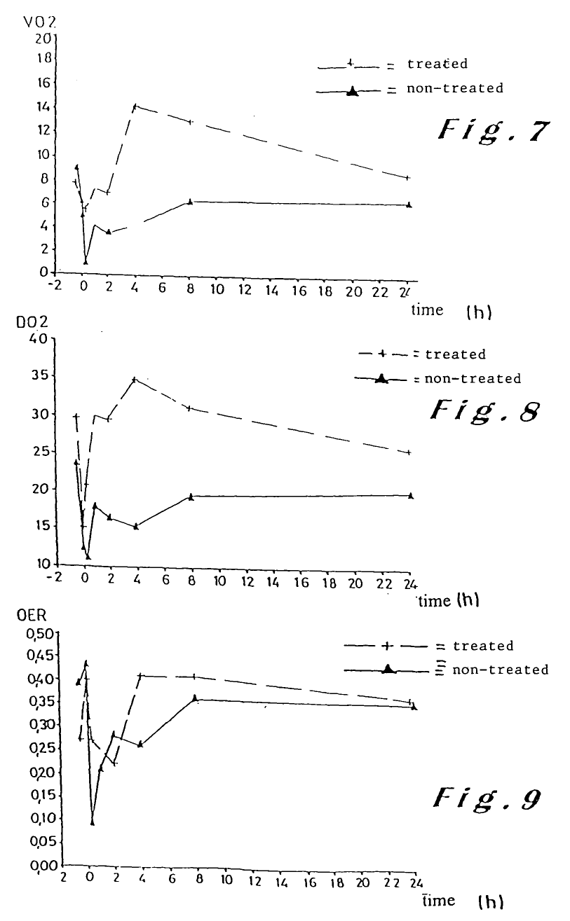

As shown by FIG. 7, unlike what was observed in untreated animals, the composition according to the invention makes it possible to maintain the oxygen consumption (VO₂) at its base level, or even to increase it, thus preventing the development of tissue hypoxia. This maintaining or even increase are made possible by virtue of the following mechanisms:

a maintenance of the quantity of oxygen carried to the tissues by the blood (DO₂), because in particular of the increase in the cardiac output (haemodynamic effect). The change in the DO₂ is set out in FIG. 8;

an increase in the oxygen extraction capacity at the tissue level, illustrated in FIG. 9.

This increase in the OER is explained by the same phenomena as those described in diarrhoeic calves.

### EXAMPLE 4

A comparative test was carried out in accordance with the methodology described for Example 3. During this test, a clinical score was established according to the criteria described for Example 2.

The results of this evaluation are set out in Table 2 below.

TABLE 2

| Parameters/ | Calves treated | Calves not treated |
|---|---|---|
| Time (h) | (n = 3) | (n = 3) |
| Clinical score | | |
| −0.5 | 1.0 ± 0.0 | 1.0 ± 0.0 |
| 0 | 1.3 ± 2.0 | 4.0 ± 0.6* |
| 0.25 | 4.7 ± 17* | 7.0 ± 0.3**Δ |
| 1 | 4.7 ± 1.7 | 7.7 ± 0.9*Δ |
| 2 | 3.8 ± 1.6 | 6.0 ± 0.6* |
| 4 | 2.5 ± 0.5* | 3.3 ± 0.3* |
| 8 | 1.17 ± 0.2 | 1.7 ± 0.7 |
| 24 | 1.0 ± 0.0 | 1.7 ± 0.7 |

The values are expressed as follows: averages ± SE.
*: value significantly different from the value recorded at T-30 (physiological value) in the same group (*: p < 0.05, **: p < 0.01)
Δ: value significantly different from the value recorded T0 in the same group (Δ: p < 0.05).

It must be understood that the present invention is in no way limited to the embodiments described above and that many modifications can be made thereto without departing from the scope of the accompanying claims.

It is possible for example to envisage a composition in the form of at least one solid formulation, for example a powder or a soluble or effervescent tablet, intended to form at least one aqueous perusfion solution by mixing with water. Thus it is possible easily to store the components of the composition according to the invention and to prepare the solutions to be perfused just before administration.

1. Medicinal composition comprising, in therapeutically effective quantities,
hypertonic sodium chloride,
at least one molecule directly or indirectly exerting a relaxation effect on the vascular smooth muscles, and
at least one molecule able to supply an exogenous phosphate input.

2. Composition according to claim 1, characterised in that it also comprises an alkalinising agent able to increase the blood pH in a range compatible with suitable transportation of oxygen.

3. Composition according to one or other of claims 1 and 2, characterised in that it is in the form of at least one aqueous solution.

4. Composition according to claim 3, characterised in that it consists of a combination of a first perfusion solution and a second perfusion solution,
the first perfusion solution comprising water, the said hypertonic sodium chloride and the said at least one molecule exerting a relaxation effect, and
the second perfusion solution comprising water, and the said at least one molecule able to supply an exogenous phosphate input.

5. Composition according to one or other of claims 1 and 2, characterised in that it is in the form of at least one solid formulation intended for constituting at least one aqueous perfusion solution by mixing with water.

6. Composition according to claim 4, characterised in that the hypertonic sodium chloride is at a concentration of 5% to 10% in the said first perfusion solution.

7. Composition according to any one of claims 1 to 6, characterised in that the said at least one molecule able to supply an exogenous phosphate input is chosen from amongst the group consisting of inorganic phosphates, organic phosphates or modifiers of the intra-erythrocyte diphosphoglycerate content.

8. Composition according to any one of claims 4 to 7, characterised in that the second perfusion solution also contains an alkalinising agent able to increase the blood pH in a range compatible with a suitable transportation of oxygen.

9. Composition according to any one of claims 2 to 8, characterised in that the alkalinising agent is chosen from amongst the group consisting of bicarbonates, acetates, proprionates and lactates.

10. Composition according to any one of claims 4 to 8, characterised in that the second perfusion solution also contains at least one component intended to correct the water, electrolytic or energy balance.

11. Composition according to claim 10, characterised in that the said at least one component intended to correct the balance is chosen from amongst the group consisting of sodium chloride, potassium chloride and glucose.

12. Composition according to any one of claims 1 to 11 for implementing a therapeutic or prophylactic treatment for hypoxic tissue disorders, in particular those arising during toxi-infectious pathologies, in mammals.

13. Composition according to claim 12 for use in the treatment of neonatal diarrhoea and endotoxin shock in ruminants.

14. Composition according to any one of claims 4 to 11, as a product combining the first perfusion solution and the second perfusion solution intended for simultaneous use thereof, separate or spread out in time, in a therapeutic or prophylactic treatment of hypoxic tissue disorders, in particular those arising during toxi-infectious pathologies, in mammals.

15. Use of a composition according to any one of claims 1 to 11 for manufacturing a medicine intended for the therapeutic or prophylactic treatment of hypoxic tissue disorders, in particular those arising during toxi-infectious pathologies, in mammals.
